(19) United States
(12) Patent Application Publication
(10) Pub. No.: US 2018/0194967 A1
(43) Pub. Date: Jul. 12, 2018

(54) ANTIMICROBIAL PEPTIDE AMPHIPHILE COATINGS

(71) Applicants: BRET ULERY, COLUMBIA, MO (US); ROGER DE LA TORRE, COLUMBIA, MO (US); JOSIAH SMITH, COLUMBIA, MO (US); JULIE NGUYEN, COLUMBIA, MO (US)

(72) Inventors: BRET ULERY, COLUMBIA, MO (US); ROGER DE LA TORRE, COLUMBIA, MO (US); JOSIAH SMITH, COLUMBIA, MO (US); JULIE NGUYEN, COLUMBIA, MO (US)

(21) Appl. No.: 15/867,062

(22) Filed: Jan. 10, 2018

Related U.S. Application Data
(60) Provisional application No. 62/444,540, filed on Jan. 10, 2017.

(51) Int. Cl. C09D 189/00; A61L 31/10; A61L 31/04; A61L 31/16; C09D 5/14; A01N 37/46

(52) U.S. Cl. CPC C09D 189/00; A61L 31/10; A61L 31/048; A61L 31/16; A61L 2420/02; A01N 37/46; A61L 2300/404; A61L 2300/25; C09D 5/14

(57) ABSTRACT

The present invention, in certain embodiments, is directed to a peptide amphiphile coating on the hydrophobic surface of an object, such as a medical device, in which non-covalent associations attach the hydrophobic portion of the peptide amphiphile to the hydrophobic surface. Other embodiments of the invention are directed to a method for forming the coating.

### CROSS-REFERENCE TO RELATED APPLICATIONS

This application is based on and claims priority to U.S. Provisional Application Ser. No. 62/444,540, filed on Jan. 10, 2017, which is hereby incorporated herein by reference.

### STATEMENT REGARDING FEDERALLY SPONSORED RESEARCH OR DEVELOPMENT

Not applicable.

### BACKGROUND OF THE INVENTION

### 1. Field of the Invention

The present invention is directed to the field of antimicrobial coatings, such as those that may be useful in connection with invasive medical devices.

### 2. Description of Related Art

Nosocomial (hospital-acquired) infections are a significant problem in the healthcare industry as evidenced by the fact that there were an estimated 722,000 cases of such infections, ultimately leading to approximately 75,000 deaths, in the United States in 2011. A significant portion of these infections are caused by the use of invasive medical devices such as endotracheal tubes and catheters. If the surfaces of such medical devices could be modified to prevent bacterial growth, then the incidence of nosocomial infections could be greatly decreased.

Peptides that possess antimicrobial behavior may be used to inhibit bacterial growth. However, standing alone, peptides serve as a weak coating for medical devices due to the fact that they are water soluble. As a result, the peptides may be easily washed away from the surface of a medical device.

### BRIEF SUMMARY OF THE INVENTION

Certain embodiments of the present invention are directed to a process for coating an object with a peptide amphiphile comprising dissolving a peptide amphiphile in a solvent to form a solution, wherein a concentration of the peptide amphiphile in the solution is below an upper critical micelle concentration (CMC) bound for the peptide amphiphile in the solvent; submerging at least a portion of an object having a hydrophobic surface in the solution; maintaining the portion of the object submerged in the solution for a time sufficient to form a layer of the peptide amphiphile on the hydrophobic surface of the portion of the object. In certain aspects of the invention, the maintaining step ranges from 1 minute to 2 hours.

In certain embodiments, the concentration of the peptide amphiphile in the solution is 50%, or 90%, below the upper CMC bound of the peptide amphiphile in the solvent or lower and may be maintained at 50% below the upper CMC bound or lower throughout the maintaining step. Suitable concentrations of the peptide amphiphile in the solution include from 1 μM to 1 mM, which may be maintained throughout the maintaining step. In certain aspects of the invention, the concentration of the peptide amphiphile in the solution is above a lower CMC bound of the peptide amphiphile in the solvent and in other aspects, the concentration of the peptide amphiphile in the solution is below a lower CMC bound of the peptide amphiphile in the solvent.

In certain embodiments, the peptide amphiphile layer is formed on the object prior to the formation of one or more peptide amphiphile micelles in the solution. In certain aspects of the invention, the layer is formed prior to evaporation of the solution that concentrates the peptide amphiphile in the solution above the upper CMC bound.

In certain embodiments, the peptide amphiphiles forming the layer are not part of a micelle, the layer may be substantially free of micelles, and/or the layer may be free of micelles.

In certain embodiments of the invention, a hydrophobic portion of the peptide amphiphile interacts with the hydrophobic surface of the object to form the layer. The hydrophobic portion of the peptide amphiphile may comprise a lipid selected from the group consisting of linear fatty acids, palmitic acid, lauric acid, lipids containing ring structures, mono or poly-unsaturated lipids, palmitoleic acid, hexadecylamine, and hexadecylamine-maleimide, alkyl amines, and combinations thereof.

In certain embodiments of the invention, the peptide of the peptide amphiphile is a hydrophilic antimicrobial peptide, such as AB01, SPIKE, Poly(KV), and combinations thereof.

In certain embodiments, the hydrophobic surface of the object comprises a hydrophobic polymer selected from the group consisting of polyvinylchloride (PVC), silicone, polyethylene, polystyrene, polypropylene, teflon and combinations thereof, which polymer may be a medical-grade plastic.

In some embodiments, the solvent used in the process of the invention may comprise water, saline, hexane, methanol, diethyl ether, ethanol, or combinations thereof, and may be an aqueous solution. The solution pH may be altered to affect the CMC of the peptide amphiphiles.

Certain embodiments of the invention are directed to a peptide amphiphile coating for an object having a hydrophobic surface comprising a plurality of peptide amphiphiles comprising a hydrophobic tail and a hydrophilic peptide; wherein the hydrophobic tail is non-covalently associated with the hydrophobic surface; and wherein the hydrophilic peptide is oriented away from the object. In certain aspects of the invention, the amphiphile coating has a thickness ranging from 10 to 100 nm. In certain aspects of the invention, the thickness ranges from 30 to 60 nm. In certain aspects of the invention, the contact angle of the peptide amphiphiles to the hydrophobic surface ranges from 5° to 90°, or from 50° to 100°.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 is a representation of the coating of one embodiment of the invention.

FIG. 2 shows the micelle concentration of an AB01-KPalm peptide amphiphile at various concentrations of the peptide amphiphile.

FIG. 3 shows representations of the thickness of a coating of one embodiment of the present invention.

FIG. 4A shows the results of a mammalian cell proliferation study of a coating of one embodiment of the present invention.

FIG. 4B shows the results of a mammalian cell activity study of a coating of one embodiment of the present invention.

### DETAILED DESCRIPTION OF PREFERRED EMBODIMENT

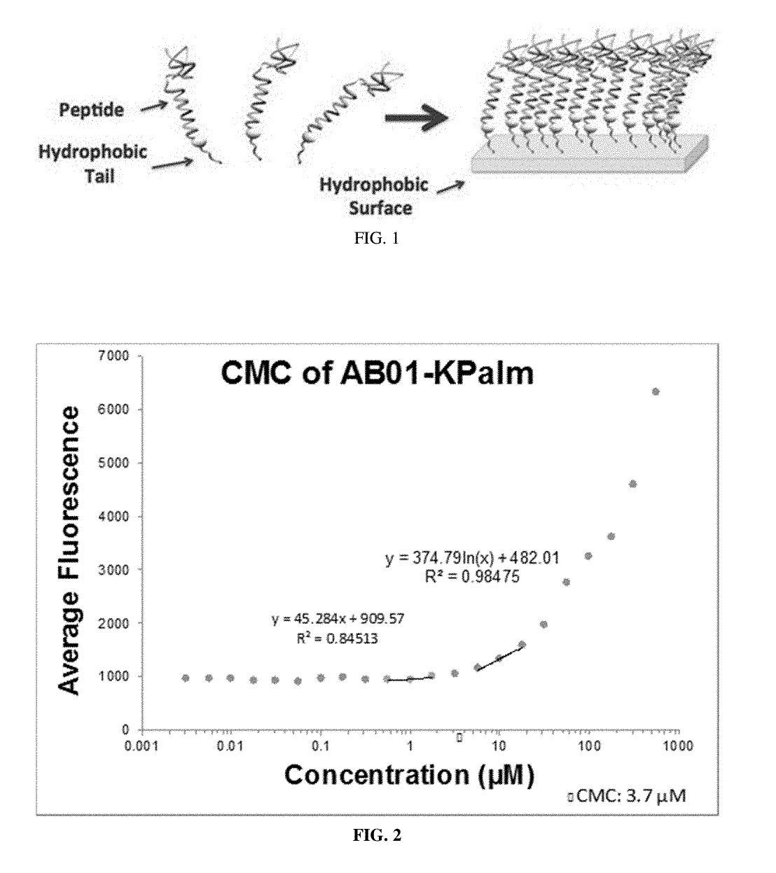

Certain aspects of present invention are directed to a process for coating an object with a peptide amphiphile. Certain other aspects are directed to a peptide amphiphile coating for an object that may be prepared via the process of the present invention, as depicted in FIG. 1.

Peptide amphiphiles are a class of molecules comprising a peptide covalently linked to a hydrophobic tail, which is usually a lipid, and most commonly a fatty acid. The peptides of the peptide amphiphiles used in the present invention are hydrophilic. As a result, the peptide amphiphiles used in the present invention are soluble in water and other solvents such that they will form aggregates, known as micelles, if the concentration of the peptide amphiphiles is high enough.

The concentration at which micelles form is called the critical micelle concentration (CMC) and occurs over a range between a lower CMC limit or bound (referred to herein as the “lower CMC bound”) and an upper CMC limit or bound (referred to herein as the “upper CMC bound”). If the concentration of the peptide amphiphiles is below the lower CMC bound, no peptide amphiphile micelles will form. If the concentration of the peptide amphiphiles is between the lower CMC bound and upper CMC bound, some (but not all) peptide amphiphiles in solution will form micelles. This lower CMC bound and upper CMC bound for a solution can be determined by measuring fluorescence, as will be readily understood by one of ordinary skill in the art. One method for determining the lower CMC bound and upper CMC bound is described in Example 1 and depicted in FIG. 2. If the concentration of peptide amphiphiles is above the upper CMC bound, all peptide amphiphiles in solution will form micelles.

Micelles form in a solution because the hydrophobic tails of the peptide amphiphiles segregate themselves from the solvent by associating with each other to form hydrophobic tail cores, which in turn displace the solvent. As a result, the corona of each micelle will display the peptides of the peptide amphiphiles. The interaction of the hydrophobic tails with each other is a form of noncovalent hydrophobic interaction, such as Van der Waals forces. The tail used can affect the stability of the resulting micelle and the lower and upper CMC bounds. The present invention leverages the hydrophobic interactions of peptide amphiphiles to deposit peptide amphiphiles as a layer, or coating, on hydrophobic surfaces of objects, where the hydrophobic tail of the peptide amphiphile interacts with the hydrophobic surface to “attach” the amphiphile to the surface.

In the process of the present invention, the concentration of the peptide amphiphiles in the solution is maintained at a level at which the hydrophobic tails of the peptide amphiphiles in the solution will associate with the hydrophobic surface of the object. This concentration is below the upper CMC bound so that all of the peptide amphiphiles do not form micelles. Preferably the concentration of peptide amphiphiles is sufficiently below the upper CMC bound such that the hydrophobic interactions between peptide amphiphiles is not sufficient to form a significant micelle population that would be disruptive to the process of forming the peptide amphiphile coating. In certain embodiments, the concentration of peptide amphiphiles is at a level at which the peptide amphiphiles preferentially assemble as a coating on the object over forming micelles in the solution.

The peptide amphiphile coating of the present invention is resistant to dissolving in water or other aqueous environments. This resistance is due to the hydrophobic interactions between the hydrophobic tails of the peptide amphiphiles and the hydrophobic surface of the objects on which they are assembled. Because the hydrophobic tails are associated with the surface of the object, the peptides of the peptide amphiphiles are displayed externally on the surface of the object, which imparts antimicrobial benefits to the objects to which the coatings are applied.

Certain embodiments of the invention are directed to a process that includes the steps of: 1) dissolving a peptide amphiphile in a solvent to form a solution, wherein the concentration of the peptide amphiphile in the solution is below the upper CMC bound, 2) submerging at least a portion of an object having a hydrophobic surface in the solvent, and 3) maintaining the portion of the object submerged in the solvent for a time sufficient to form a layer of the peptide amphiphile on the hydrophobic surface of the portion of the object.

During formation of the coating of the present invention, the concentration of the peptide amphiphile in the solution is below the upper CMC bound. At or above the upper CMC bound, the peptide amphiphiles will aggregate to form micelles instead of coating the object. A concentration below the upper CMC bound will allow a layer of peptide amphiphiles to form on the object. The concentration of the peptide amphiphiles in the solution may be on the spectrum from the upper CMC bound to the lower CMC bound, such that even though there are enough hydrophobic interactions for the peptide amphiphiles to aggregate into micelles, the concentration is low enough that the peptide amphiphiles will form a layer on the hydrophobic plastic if the object is submerged for a sufficient length of time. In certain embodiments, the concentration of the peptide amphiphiles is below the lower CMC bound. In such embodiments, even though there are fewer hydrophobic forces to attract the peptide amphiphiles to the surface of the object, a coating will form if the object is maintained in the solution for a sufficient period of time.

In some embodiments, the concentration of the peptide amphiphile in the solution is at least 1%, 5%, 10%, 15%, 20%, 25%, 30%, 35%, 40%, 45%, 50%, 55%, 60%, 65%, 70%, 75%, 80%, 85%, 90%, 95%, (wt/vol), or any value therebetween, below the upper CMC bound of the peptide amphiphile in the solution, In certain embodiments, the concentration is at least 50%, preferably at least 75%, more preferably at least 90% (wt/vol) below the upper CMC bound. In one exemplary embodiment, described in Example 1, below, the lower CMC bound is 0.01 mg/mL, the upper CMC bound is 5 mg/ml and the concentration of peptide amphiphile used in the process of the invention was 0.1 mg/mL, or 98% below the upper CMC bound. In certain embodiments, the concentration of the peptide amphiphile in the solution is from 1 μM to 1 mM. In certain other embodiments the concentration of the peptide amphiphile in the solution is from 0.01 mg/mL and 5.0 mg/mL, from 0.05 to 3.0 mg/mL, from 0.1 to 2.0 mg/mL, or any ranges therebetween. In certain embodiments, the solution pH is altered to affect the CMC concentration of the peptide amphiphile. Changes to the solution pH affect the hydrophobic surface amphiphile associations, resulting in changes to the resulting coating

Although some evaporation may occur while the object is submerged and the layer is being formed, the evaporation should not be sufficient to increase the concentration above the upper CMC bound until the desired layer is formed. It should be understood that the layer does not need to completely coat the surface, although it may. The concentration is preferably maintained below the target concentrations discussed in the previous paragraph until the desired layer is formed. Preferably the concentration of the peptide amphiphile does not rise more than 20%, preferably not more than 10%, and more preferably not more than 5% while the desired layer is being formed. In some embodiments, the concentration of the peptide amphiphile is maintained between the upper CMC bound and the lower CMC bound of the peptide amphiphile in the solution throughout the maintaining step, and may be maintained from 5% to 99.9% below, from 75% to 99% below, from 60% to 98% below the upper CMC bound, or any ranges therebetween. In certain embodiments, the concentration is maintained from 50% to 99.9% below, from 80 to 99.0% below, or from 95 to 98% below the upper CMC bound.

If the peptide amphiphile concentration is maintained below the lower CMC bound, the peptide amphiphile layer will form prior to the formation of one or more peptide amphiphile micelles. If the concentration is above the lower CMC bound the concentration is preferably maintained such that the peptide amphiphiles forming the peptide amphiphile layer are not part of a micelle. However, it is possible that some micelles will be contained in the layers as it forms. In certain embodiments, the layer is preferably substantially free of micelles and may be free of micelles. If micelles are present within the layer, they preferably form less than 50% (wt/wt) of the layer, less than 25% (wt/wt) of the layer, less than 5% (wt/wt) of the layer, or any percentage therebetween. In certain embodiments, once the layer forming is complete, the solvent is allowed to evaporate. Although micelles will form at this stage if the amphiphiles have not all assembled on into the layer that forms the coating, the coating is already formed at that point. Micelles may form on top of the coating, but such micelles are not considered to be present within the layer.

The peptide of the peptide amphiphile may be any hydrophilic antimicrobial peptide. If a peptide is too hydrophobic, then the peptide amphiphile will not form micelles. In certain embodiments, the peptides are small peptides. In some such embodiments, the peptides may have 50 or fewer amino acids, 25 or fewer amino acids, or 10 or few amino acids, or any number of amino acids therebetween. In certain embodiments, the peptides have either a net neutral or net positive charge. Suitable hydrophilic antimicrobial peptides include, but are not limited to, AB01 (a net-positively charged cyclic peptide designed to penetrate biofilms and disrupt bacteria membranes), SPIKE (a peptide containing positive and negatively charged amino acids designed to puncture bacteria membranes), and Poly(KV), preferably Poly(K₂₀V₁₀), (a peptide consisting of combinations of a positively charged amino acid such as lysine alternating randomly with valine or another non-charged amino acid which can be produced in via n-carboxyanhydride ring-opening polymerization derived from a similar polymer shown to be antibacterial in the literature), and combinations thereof.

The hydrophobic tail of the peptide amphiphile serves to interact with the hydrophobic surface of the object to which the peptide amphiphile coating is applied. Suitable hydrophobic tails of the peptide amphiphile include, but are not limited to lipids, fatty acids, linear fatty acids, palmitic acid, lauric acid, lipids containing ring structures, mono or poly-unsaturated lipids, palmitoleic acid, hexadecylamine, and hexadecylamine-maleimide, alkyl amines and combinations thereof.

Suitable peptide amphiphiles include AB01-KPalm [FRIRVRV[DRR-dNal(2′)-FWRK]dV-dP [SEQ. ID. NO. 1]-K(Palm)], C₁₆Mal_C(K₄/E₄)-SPIKE, [C₁₆-Mal_C-E₄/K₄-NQVFLFKDDKYWLISN [SEQ. ID. NO. 2]], preferably containing a mixture of the K₄ and E₄ versions at a 1:1 molar ratio, and Poly(K₂₀V₁₀)-C₁₆.

The object may be any object that has a hydrophobic surface. All or part of the surface may be hydrophobic. The object may be formed from the hydrophobic material or may be coated with the hydrophobic material. The object is preferably a medical device, preferably an invasive medical device for implantation or other interaction with the body, and the hydrophobic surface may comprise a medical grade polymer or plastic. Because medical devices are commonly composed of moderately hydrophobic polymers such as polyvinylchloride (PVC) and silicone, the process and coating of the present invention can be used with many exiting medical devices. Exemplary objects made of medical grade plastic include, but are not limited to, endotracheal tubes and Foley catheters. The hydrophobic surface of the object may include a hydrophobic polymer. The hydrophobic polymer must be stable in the solution. Suitable polymers include, but are not limited to, polyvinylchloride (PVC), silicone, polyethylene, polystyrene, polypropylene, teflon, or combinations thereof.

Suitable solvents are ones in which a portion of the peptide amphiphile is insoluble and another portion is soluble, allowing for aggregation. Suitable solvents enable the self-assembly of the peptide amphiphiles and are compatible with the peptide amphiphile and hydrophobic polymer surface. Suitable solvents include, but are not necessarily limited to, water, saline, hexane, methanol, diethyl ether, ethanol, or combinations thereof. In one embodiment, the solvent is an aqueous solution.

At least a portion of the object having a hydrophobic surface is submerged in the solution. If only a portion of the surface of the object is hydrophobic, the portion that is submerged will include at least a portion of the hydrophobic surface.

As discussed above, the object is maintained in the solution for a time sufficient to form a layer of the peptide amphiphile on the hydrophobic surface of the portion of the object. The layer may completely coat the submerged hydrophobic surface or may coat only a portion of the surface. Preferably the layer coats at least 50%, at least 75% at least 90%, 100% or any value therebetween, of the submerged hydrophobic surface. The object is preferably maintained in the solution for a time period sufficient to form the desired coverage and thickness of the peptide amphiphile layer, preferably ranging from 1 minute to 5 hours, from 5 minutes to 2 hours, or from 1 to 30 minutes, and any time intervals therebetween. The solution may be maintained at room temperature using aseptic conditions.

The peptide amphiphile coating of the invention may be formed by the process of the invention described above, or by other processes that will form a layer of peptide amphiphiles assembled on a hydrophobic surface by the interactions between the hydrophobic tails of the amphiphiles and the hydrophobic surface. The peptide amphiphile coating of the invention may include any of the aspects of the peptide amphiphile layer and coating discussed above.

The peptide amphiphile coating of the instant invention comprises a plurality of peptide amphiphiles having a hydrophobic tail at one end and a hydrophilic peptide at the opposite end. The hydrophobic tail of the peptide amphiphiles are non-covalently bound to the hydrophobic surface, thereby attaching the amphiphile to the surface. The hydrophilic peptide is in turn oriented away from the object. The coating preferably comprises a homogeneous layer of the peptide amphiphiles.

Suitable hydrophobic tails include, but are not necessarily limited to those discussed above with respect to the process of the invention. Suitable hydrophilic peptides include the hydrophilic antimicrobial peptides discussed above with respect to the process of the invention.

The coating has a thickness of peptide amphiphiles ranging preferably from 10 nm to 80 nm, more preferably from 20 nm to 70 nm, and most preferably from 30 nm to 60 nm, although it can be any range or thickness therebetween.

The contact angle of the peptide amphiphiles to the surface preferably ranges from 5° to 120°, more preferably from 50° to 100°, and most preferably from 70° to 100°, or any ranges therebetween.

As discussed above, the coating of the present invention does not readily dissolve in water due to the noncovalent associations between the hydrophobic tails of the peptide amphiphiles and the hydrophobic surface of the object. This is a benefit over known techniques in which micelles may be deposited on a surface by evaporation, wherein the micelles readily dissolve when exposed to aqueous environment. Because the antimicrobial peptides are oriented away from the surface, the peptides will be exposed to the body or other environment in which they are used, where they serve to prevent or reduce bacterial colonization and subsequent hospital-acquired (nosocomial infections). This imparts lasting antimicrobial benefits to the objects on which the coating is formed.

### Example 1

Peptide Amphiphiles

Three antimicrobial peptide amphiphiles were synthesized. The first, AB01-KPalm [FRIRVRV[DRR-dNal(2′)-FWRK]dV-Dp [SEQ. ID. NO. 1]-K(Palm)] is an antibacterial and antibiofilm peptide amphiphile with K(Palm) (palmitoyllysine). Second, C₁₆Mal_C(K₄/E₄)-SPIKE [C₁₆-Mal_C-EEEE/KKKK-NQVFLFKDDKYWLISN [SEQ. ID. NO. 2]] is a novel peptide amphiphile derived from SPIKE, a peptide shown to be antibacterial in the literature. The SPIKE peptide amphiphile was used as a mixture of the K₄ and E₄ versions at a 1:1 molar ratio. Third, Poly(K₂₀V₁₀)-C₁₆ is a peptide polymer produced via n-carboxyanhydride ring-opening polymerization derived from a similar polymer shown to be antibacterial in the literature.

Peptide Amphiphile Concentration in the Solution

The critical micelle concentration of AB01-KPalm was assessed. AB01-KPalm was dissolved in an aqueous solution containing 1 μM diphenylhexatriene (DPH). Hydrophobic DPH will sequester within the hydrophobic micelle core and undergoes a dramatic increase in fluorescence when sequestered due to molecular stacking. This fluorescence increase indicates the peptide amphiphile (“AMPA”) molecules are of sufficient concentration to aggregate, forming micelles, which is the concentration at the lower CMC bound. This continues with increasing AMPA concentration indicating the presence of a larger population of micelles in solution. The CMC was found to be 3.7 μM or about 0.01 mg/mL (FIG. 2). Submersion coating significantly above this concentration (at 0.1 mg/mL) assures that sufficient AMPAs are present to drive the assembly of AMPAs into a coating on the object. For AB01-KPalm, the concentration range appropriate for submersion coating is from about 0.01 mg/mL at the lower CMC bound to about 5 mg/mL at the upper CMC bound, which is the approximate aqueous solubility limit of this AMPA. This range can be determined experimentally for other peptide amphiphiles.

Submersion of Devices

AMPAs were dissolved in a 10 mM NaCl aqueous solution at room temperature (˜22° C.) and vortexed for 10 minutes within one week of anticipated use. All AMPAs were diluted to working concentrations from 10 mg/mL or 1 mg/mL stock solutions immediately prior to submersion coating experiments. A concentration of 1 mg/mL was used for Poly(K₂₀V₁₀)-C₁₆ and 0.1 mg/mL for AB01-KPalm and C₁₆Mal_C(K₄/E₄)-SPIKE. Submersion coating was completed using either 1.0 or 2.0 mL of coating solution in a 7 or 15 mL glass vial, respectively. The coating process was completed under aseptic conditions.

Coated Devices

Sections of polyvinylchloride (PVC) endotracheal tubes (ETT) with a surface area of about 1 cm² were removed from a new, whole ETT using a hole punch. Silicone, the polymer catheters are commonly composed of, was processed into sections using the same method as ETTs. ETT and silicone cutouts were submersion coated using 1 mL AMPA solutions.

For surface topology studies, PVC powder was purchased and dissolved in 3:2 cyclopentanone:tetrahydrofuran at a concentration of 10 mg/mL. PVC solution (50 μL) was used to spin-coat 1 cm diameter glass slides for 1 minute at 3,000 RPM. The PVC-coated slides were allowed to dry for 24 hours prior to submersion coating in 2 mL AMPA solutions.

Time for Coating

Submersion coating was completed by suspending ETT sections, catheter sections, or PVC-coated glass slides in the desired solution for 5 minutes to 2 hours.

Temperature for Coating

All studies were completed at room temperature (about 22° C.).

Coating Characterization

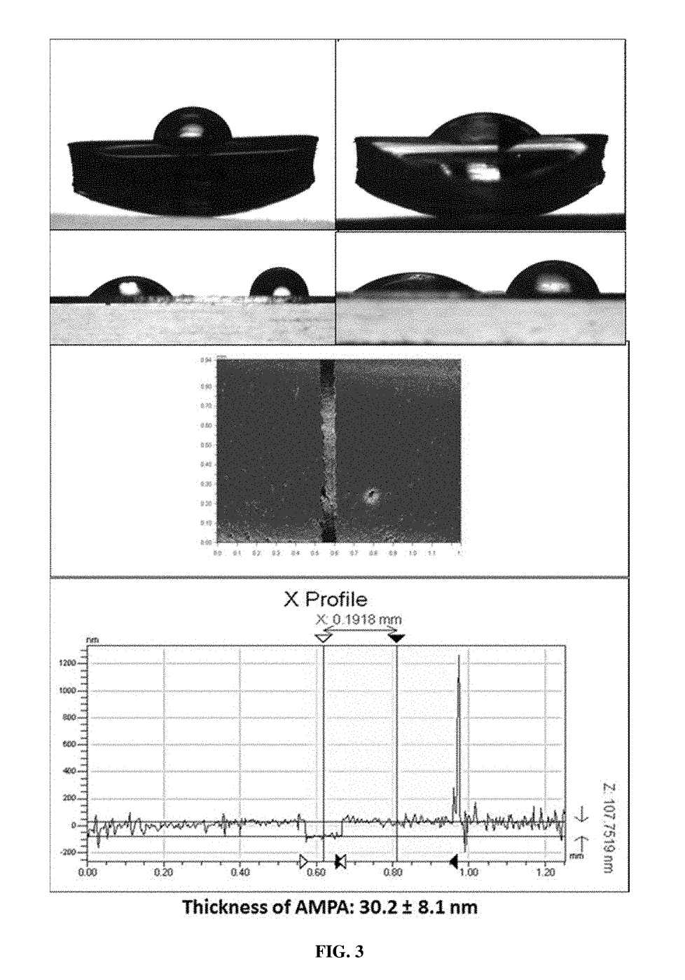

Coated products were removed from submersion and dried in 24-well plates under aseptic conditions for at least 24 hours prior to characterization. The coating was assessed via contact angle to evaluate surface hydrophilicity and optical profilometry to measure coating thickness. FIG. 3 shows that an AB01-KPalm-coating dramatically changes the hydrophilicity of PVC ETTs (top: left—uncoated; right—AB01-KPalm coated) and PVC-coated glass slides (second line: left—glass/PVC coated glass; right—glass/PVC and AMPA coated glass) and does so by a very thin coating of approximately 30 nm (third line: AMPA and PVC thickness on glass slide; fourth line: measurement of coating thickness). Optical profilometry shows the coating has a consistent morphology and completely covers the surface. Electron microscopy of the AMPA coatings has shown no presence of micelles.

Toxicity Studies

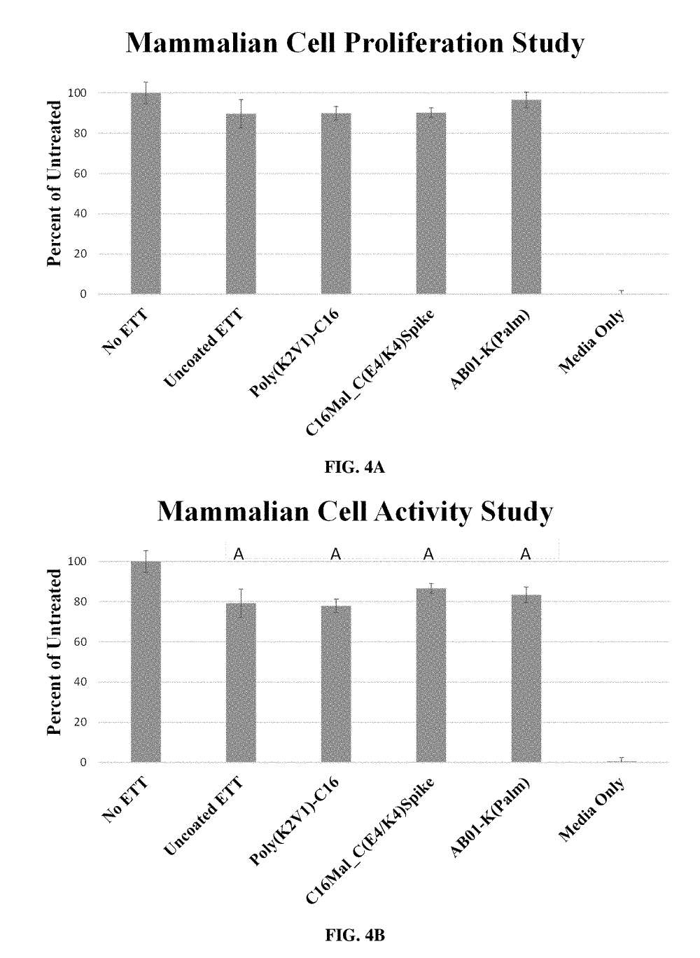

The AMPA coatings showed no in vitro toxicity against mammalian cells. Murine endothelial cells were cultured for 72 hours in the presence of uncoated or AMPA-coated ETTs. The proliferation study assessed the number of cells after the incubation period using the PicoGreen assay and the cell activity evaluated the health of these cells using a MTS assay. No statistical difference was found with any coating compared to uncoated ETTs via a Tukey-Kramer statistical analysis (α=0.05) as shown in FIG. 4A and FIG. 4B. This data suggests that the AMPA coatings are non-toxic to mammalian cells.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A process for coating an object with a peptide amphiphile comprising:
dissolving a peptide amphiphile in a solvent to form a solution, wherein a concentration of the peptide amphiphile in the solution is below an upper critical micelle concentration (CMC) bound for the peptide amphiphile in the solvent;
submerging at least a portion of an object having a hydrophobic surface in the solution; and
maintaining the portion of the object submerged in the solution for a time sufficient to form a layer of the peptide amphiphile on the hydrophobic surface of the portion of the object.

2. The process of claim 1, wherein the concentration of the peptide amphiphile in the solution is 50% below the upper CMC bound of the peptide amphiphile in the solvent or lower.

3. The process of claim 2, wherein the concentration of the peptide amphiphile in the solution is 90% below the upper CMC bound of the peptide amphiphile in the solvent or lower.

4. The process of claim 2, wherein the concentration of the peptide amphiphile in the solution is maintained at 50% below the upper CMC bound or lower throughout the maintaining step.

5. The process of claim 1, wherein the concentration of the peptide amphiphile in the solution is from 1 μM to 1 mM throughout the maintaining step.

6. The process of claim 1, wherein the concentration of the peptide amphiphile in the solution is above a lower CMC bound of the peptide amphiphile in the solvent.

7. The process of claim 1, wherein the concentration of the peptide amphiphile in the solution is below a lower CMC bound of the peptide amphiphile in the solvent.

8. The process of claim 1, wherein the layer is formed prior to the formation of one or more peptide amphiphile micelles.

9. The process of claim 1, wherein the layer is formed prior to evaporation of the solution that concentrates the peptide amphiphile in the solution above the upper CMC bound.

10. The process of claim 1, wherein the peptide amphiphiles forming the layer are not part of a micelle.

11. The process of claim 1, wherein the layer is substantially free of micelles.

12. The process of claim 11, wherein the layer is free of micelles.

13. The process of claim 1, wherein a hydrophobic portion of the peptide amphiphile interacts with the hydrophobic surface of the object to form the layer.

14. The process of claim 1, wherein the peptide amphiphile comprises a lipid selected from the group consisting of linear fatty acids, palmitic acid, lauric acid, lipids containing ring structures, mono or poly-unsaturated lipids, palmitoleic acid, hexadecylamine, hexadecylamine-maleimide, linear alkyl amines, and combinations thereof.

15. The process of claim 1, where the solvent comprises water, saline, hexane, methanol, diethyl ether, ethanol, or combinations thereof.

16. The process of claim 15, wherein the solution is an aqueous solution.

17. The process of claim 1, wherein the hydrophobic surface of the object comprises a hydrophobic polymer selected from the group consisting of polyvinylchloride (PVC), silicone, polyethylene, polystyrene, polypropylene, teflon and combinations thereof.

18. The process of claim 17, wherein the polymer is a medical-grade plastic.

19. The process of claim 1, wherein a peptide of the peptide amphiphile is a hydrophilic antimicrobial peptide.

20. The process of claim 19, wherein the hydrophilic antimicrobial peptide is selected from the group consisting of AB01, SPIKE, Poly(KV), and combinations thereof.

21. The process of claim 1, wherein the maintaining step is from 1 minute to 2 hours.

22. The process of claim 1, wherein a solution pH is altered to affect the CMC of the peptide amphiphiles.

23. The product of the process of claim 1.

24. A peptide amphiphile coating for an object having a hydrophobic surface comprising:
a plurality of peptide amphiphiles comprising a hydrophobic tail and a hydrophilic peptide;
wherein the hydrophobic tail is non-covalently bound to the hydrophobic surface; and
wherein the hydrophilic peptide is oriented away from the object.

25. The peptide amphiphile coating of claim 24, wherein the coating has a thickness ranging from 10 to 100 nm.

26. The peptide amphiphile coating of claim 25, wherein the thickness ranges from 30 to 60 nm.

27. The peptide amphiphile coating of claim 24, wherein the contact angle of the peptide amphiphiles to the hydrophobic surface ranges from 5° to 120°.

28. The peptide amphiphile coating of claim 27, wherein the contact angle ranges from 50° to 100°.


SEQUENCE LISTING (3 sequences)

SEQ ID NO: 1 - protein, 18 aa, Artificial Sequence
  Synthetic
  MISC_FEATURE (8)..(15): Cyclic portion of the peptide; terminal amino acid side chains are covalently linked (D to K)
  MISC_FEATURE (11)..(11): d enantiomer of 2-naphthylalanine
  MISC_FEATURE (16)..(16): d enantiomer
  MISC_FEATURE (17)..(17): d enantiomer
  MISC_FEATURE (18)..(18): palmitollysine
FRIRVRVDRRXFWRKVPK

SEQ ID NO: 2 - protein, 21 aa, Artificial Sequence
  Synthetic
  MISC_FEATURE (1)..(1): 1-Hexadecyl-1H-pyrrole-2,5-dione covalently bond to the side-chain of cysteine
CEEEENQVFLFKDDKYWLISN

SEQ ID NO: 3 - protein, 21 aa, Artificial Sequence
  Synthetic
  MISC_FEATURE (1)..(1): 1-Hexadecyl-1H-pyrrole-2,5-dione covalently bond to the side-chain of cysteine
CKKKKNQVFLFKDDKYWLISN